(19) United States
(12) Patent Application Publication
(10) Pub. No.: US 2009/0204120 A1
(43) Pub. Date: Aug. 13, 2009

(54) Bone Cement Application System

(75) Inventors: Volker Trösken, Witten (DE); Martin Deli, Mülheim (DE); Jürgen Speder, Bochum (DE); Katja Gonschorek, Bochum (DE); Ronald Kontek, Herne (DE); Marion Denk, Bochum (DE); Dietrich Grönemeyer, Sprockhövel (DE)

Correspondence Address: REED SMITH LLP, 3110 FAIRVIEW PARK DRIVE, SUITE 1400, FALLS CHURCH, VA, 22042, US

(73) Assignee: AMEDO GMBH, BOCHUM (DE)

(21) Appl. No.: 11/992,390

(22) PCT Filed: Sep. 13, 2006
(86) PCT No.: PCT/EP2006/008897

§ 371 Date: Mar. 25, 2009

(30) Foreign Application Priority Data
Sep. 22, 2005 (DE) 10 2005 045 227.2

(51) Int. Cl. A61B 17/58

(52) U.S. Cl. 606/93; 606/92

(57) ABSTRACT

The invention relates to a device for the application of bone cement, in particular in the field of vertebroplasty, comprising an application conduit (1) guiding the bone cement towards the application area and a flexible piston rod (2) longitudinally movable in the application conduit (1) and provided with a piston (3) arranged at the distal end of the rod (2), said piston being in contact with and providing a seal at the internal wall of the application conduit (1), with the piston rod (2) protruding proximally from said application conduit (1) and the application conduit (1), at least partially, being formed into one or several windings (5). A spirally or helically wound application conduit (1) results in the effective useful length of the application conduit (1) being relatively great with the overall dimensions of the application device on the other hand still being moderate. Providing a long application conduit (1) enables the application conduit (1) to convey a large bone cement volume and at the same time be still of thin configuration so that the forces necessary to produce a certain pressure are held relatively low. This is advantageous in that the application device enables the attending physician to obtain significantly improved feedback during treatment.

The invention relates to a device for the application of bone cement, in particular in the field of vertebroplasty, comprising an application conduit guiding the bone cement towards the application area and a flexible piston rod longitudinally movable in the application conduit and provided with a piston arranged at the distal end of the rod, said piston being in contact with and providing a seal at the internal wall of the application conduit, with the piston rod protruding proximally from said application conduit.

Vertebroplasty still is a comparatively new treatment procedure by means of which especially diseases of the spinal column or other bones can be dealt with. Pain is frequently caused as a result of a significant loss of bone mass as, for example, encountered in diseases such as osteoporosis. In order to compensate the loss of bone mass relatively viscous bone cement is injected in vertebroplasty (or osteoplasty). Vertebroplasty may, in particular, be effectively applied for the stabilization of fractured vertebral bodies, for which purpose a bone cement is injected under pressure into the interior of the porous vertebra to quasi connect or join the individual portions of the vertebral body.

Filling the vertebrae is usually effected under radiographic control with the vertebral body or bone to be treated being punctured with the help of a vertebroplasty needle. The bone cement material used usually consists of polymethylmethacrylate (PMMA), calcium phosphate or similar materials which are prepared immediately before application and then injected into the vertebral body.

The injection is performed by means of a vertebroplasty needle to which several syringes filled with approx. 1 ml of bone cement are connected one after another.

Since bone cement is of highly viscous consistency and subject to a hardening process which, moreover, commences within a few minutes other application devices are known from prior art by means of which relatively high pressures can be achieved. Such an application device customarily comprises an application conduit through which bone cement is transferred towards the application area, a piston longitudinally movable within the application conduit so that bone cement can be sucked into the device when the piston is moved in proximal direction while movement of the piston in distal direction causes bone cement to be ejected from the application device. Such a device is known from DE 100 64 202 A1 which provides for the piston to be directly moved forward initially until a certain resistance is encountered. Following this, the application device actuation changes in that the forward movement of the piston then being achieved by a screwing motion which enables a higher pressure to be exerted on the piston and the bone cement present within the application conduit.

However, an unfavorable aspect of this approach is that the attending physician performing the screwing motion only receives insufficient feedback with respect to the accurate dosing of the bone cement. In the event complications are experienced during treatment it may well be that the physician does not notice this in time so that large amounts of bone cement are allowed to enter the vertebral body. Furthermore, even when physician has stopped the screwing motion advancing the piston a certain amount of bone cement may still continue to be ejected. Turning the piston back takes some time and, for that reason, may not be effected in good time. This drawback is of great significance, especially if leakage occurs during the treatment that goes unobserved by the physician as a result of the poor feedback and monitoring capability.

Although it is basically possible to use thin-bodied bone cement so that lower forces need be exerted, this is, however, associated with the risk that easily flowing bone cement may enter the spinal space, the nerve root spaces and even the venous plexus of the vertebral body.

To overcome these problems an injection pump has been disclosed in DE 103 53 919 A1 that is provided with a long pump body having a small surface area volume so that the force to be applied to eject the highly viscous bone cement from the distal opening is lower. For this reason the above described screwing motion is no longer needed and the piston can be moved forward and back by means of a partly rigid, partly flexible rod attached to the piston. The injection pump dealt with in that publication functions according to the simple physical relationship p=F/A, i.e. pressure=force/surface area. Reducing the cross sectional area of the injection pump thus enables sufficiently high pressures to be achieved with only smaller forces having to be exerted.

Nevertheless, the injection pump described in the latter publication also has a number of disadvantages. For instance, the amount of bone cement that can be injected by means of such an application device is relatively small, that is to say in the range of not more than 4 ml. However, since vertebral bodies are capable is of accommodating up to 10 ml the procedure requires that the application device either has to be refilled repeatedly or several application devices must be used to completely fill the vertebral body to be treated. Bearing in mind that hardening of bone cement starts six to seven minutes after application the physician must work under considerable pressure of time which may result in vertebrae being inadequately filled.

Although it may in principle be feasible to increase the volume of bone cement by providing for the entire application device to be of greater length this would make it necessary for the physician to be positioned relatively far away from the patient so that handling of such a long application device becomes difficult and the feedback the physician experiences deteriorates.

Proceeding from what is known from prior art as described hereinbefore it is therefore the objective of the invention to provide an application device that overcomes the drawbacks associated with the state of the art, enables an injection of bone cement to be performed by exerting relatively low forces and at the same time provides good feedback for the physician and has a high bone cement capacity.

According to the invention this objective is reached by a device for the application of a bone cement, in particular in the field of vertebroplasty, comprising an application conduit guiding the bone cement towards the application area and a flexible piston rod longitudinally movable in the application conduit and provided with a piston arranged at the distal end of the rod, said piston being in contact with and providing a seal at the internal wall of the application conduit, with the piston rod protruding proximally from said application conduit and the application conduit, at least partially, being formed into one or several windings.

Through the helically or spirally shaped configuration of the application conduit a system is made available in which, on the one hand, the application conduit has a great useful inner length so that, despite of its relatively narrow shaping, it is, on the other hand, capable of transferring an adequate amount of bone cement which is sufficient to fill the vertebral body completely. As a result of the relatively small diameter of the entire application conduit favorable pressure conditions are created which permits bone cement to be injected with relatively low forces having to be exerted. Viewed from the outside the entire application device at the same time is of sufficiently short length so that there will be no handling problems and the physician is positioned sufficiently close to the patient to enable good feedback results to be attained. Moreover, the wound configuration of the application conduit enables the length of the conduit to be extended so that the device requires little space only.

Since the force to be exerted is relatively low the application device is also suited for one-hand operation which facilitates handling considerably. Using the inventive application device warrants that the distance between patient and attending physician during treatment is, on the one hand, sufficiently close such that the application device can be manipulated without difficulty, but, on the other hand, also makes sure the distance to the patient is still great enough. The latter is an important requirement because filling of the vertebral body is usually effected under radiographic control which requires a certain distance to be maintained between patient and physician to prevent the physician's hands from getting into the path of the rays when the feeding system is actuated.

The portion of the flexible piston rod protruding from the application conduit may advantageously be wound up on a reel. This reel may have a cylindrical shape, for example. In this way the system design makes sure the entire application device after the application conduit is not too long and difficult to be handled. If thought expedient the wound-up flexible piston rod on the reel may be used to move the piston rod and in this way the piston arranged at the distal end of the piston rod forward and backward by turning the reel as necessary. By retracting the piston bone cement is sucked into the application conduit, moving the piston forward causes bone cement to be discharged from the distal end of the application device. To enable the reel to be turned it may be provided with a type of crank or similar arrangement.

As per an advantageous embodiment the piston rod is moved forward and/or backward by means of a ratchet mechanism. Such a ratchet mechanism is basically known, for instance as described in the German utility model DE 201 18 100 U1, and is utilized in a similar manner also for discharging sealant material from cartridges, e.g. containing silicone. Such a ratchet mechanism is provided with a piston rod of which at least some portion is serrated, with a handle being arranged on the application device by means of which a pawl is operated which causes the serrated piston rod to be moved in distal direction when the handle is actuated. If thought expedient and as described in publication DE 201 18 100 U1 a pawl preventing the piston from being retracted may be omitted so that a rapid pressure relief can be achieved when necessary.

Instead of manipulating the piston rod manually the forward and/or backward movement may also be performed by means of a motor. For example, to suck bone cement into the application device a small electric motor may be arranged at the reel onto which the flexible piston rod is wound. To discharge material from the application device a motor operation is to be viewed less favorable because in the interest of controllability this process should better be performed by the physician manually.

Advantageously, the application conduit has an inside diameter ranging between 2 to 5 mm, especially preferred is 2.5 to 3 mm. Such a diameter is considerably lower than the diameters of application devices commercially available at the present time. As a result of the small diameter the above described favorable pressure ratio is achieved so that relatively low forces need be exerted to enable a sufficiently high pressure to be produced which is necessary to enable the bone structure to be penetrated. Commercially available application devices usually have a significantly greater inside diameter requiring considerable forces to be applied to build up the required pressure, said forces to some extent can only be exerted by arranging for a screwing motion to be carried out.

As mentioned earlier, through the application device according to the invention the useful volume of the application conduit shall be increased to such an extent that the entire amount of bone cement required can be injected into the vertebral body or bone at one go. To achieve this requirement the useful volume of the application device must be in the range of about 4 to 10 ml. Typically, the useful volume is approximately 7 ml. In this way, it will not be necessary during the treatment to repeatedly refill the application device with bone cement or connect several application devices to the vertebroplasty needle one after the other. Such a procedure which is still frequently applied nowadays is associated with major disadvantages in that the attending physician acts under enormous pressure of time and still must work with highest precision because bone cement hardening commences after a few minutes. Using the application device according to the invention enables a one-time filling of the application conduit with bone cement to be achieved so that the vertebral body can be completely filled in a single treatment step.

The length of the application conduit must be selected to suit the desired useful volume and the envisaged inner diameter of the application conduit. For example, a useful application conduit length of approx. 1 m must be provided at an inside diameter of 3 mm if a volume of 7 ml is to be obtained, and it must also be taken into account in this context that not the entire length of the application conduit can be filled with bone cement because the rod with the piston attached to its distal end always projects partially into the application conduit. Taking the developed or stretched length of this application conduit the entire application device would become extremely cumbersome to handle and the physician would have to stand a considerable distance away from the patient. Due to the fact that the application conduit is formed into one or several windings, in particular wound up helically or spirally, the total dimensions of the application device can be kept within reasonable limits.

At its distal end the application conduit should be provided with connection capability for a cannula or needle by means of which the vertebra to be treated is punctured, for example, when treatment commences. Basically, such vertebroplasty needles are sufficiently known from the state of the art. In the design of said connection capability it is of great significance that the respective connection is pressure tight to make sure no leakage can occur in view of the relatively high pressures arising at the connection. For the purpose of taking in bone cement a suitable connecting element, a grommet for example, may expediently be arranged at the distal end, said grommet being immersed into the viscous bone cement. When bone cement has been taken in the connecting element is removed and the application device connected to the vertebroplasty needle. Both the vertebroplasty needle and the connecting element for taking in the bone cement may, for instance, be attached via a Luer-Lock connector of the type frequently used in medical applications.

The application conduit as well must be designed in such a way that it withstands the pressure applied. Expediently, the outside of the conduit may be suitably reinforced, for example by means of a metal braiding. For example, the application conduit may be designed in the form of a hose having sufficient flexibility to be easily wound up helically or spirally without difficulty or, alternatively, the application conduit may, for instance, be manufactured of metal and at the outset formed such that it assumes a helical or spiral shape, and in the latter case said conduit retains this shape permanently as a result of its lack of flexibility.

As per an especially advantageous embodiment the application conduit is provided with a cooling system. This configuration offers advantages associated with the fact that the mixture of the bone cement consisting of a powder and a liquid is as a rule prepared external to the body, and the hardening or curing 30 process is temperature-dependent. The lower the temperature the slower the bone cement hardening process. Bone cements customarily used at present have a pot life, i.e. the time span between mixing of the components and gelatinization of the blend, of between approx. 3 and 18 minutes. Afterwards, the bone cement can no longer be applied. Since this time span is relatively short the attending physician must work very quickly which in view of the accuracy called for is not at all unproblematic. Especially, if problems occur during the treatment the customary pot lives are hardly adequate.

Therefore, as per the beneficial embodiment of the invention the application conduit is, by means of a cooling device, typically cooled down to a temperature ranging between approx. 5 and 12° C. which corresponds roughly to twice the pot life. Advantageously, this cooling effect may be regulated to achieve appropriate cooling results, for example in the event of higher ambient temperatures. The cooling effect may, for instance, be achieved by arranging cooling coils around the application conduit.

However, particularly preferred as cooling systems are Peltier elements of which one or several are mounted on the application conduit. The Peltier technology is very cost-efficient, maintenance-free, noiseless and compact and therefore especially suitable for the inventive application conduit.

Further elucidation of the invention is provided through the enclosed figure by way of examples.

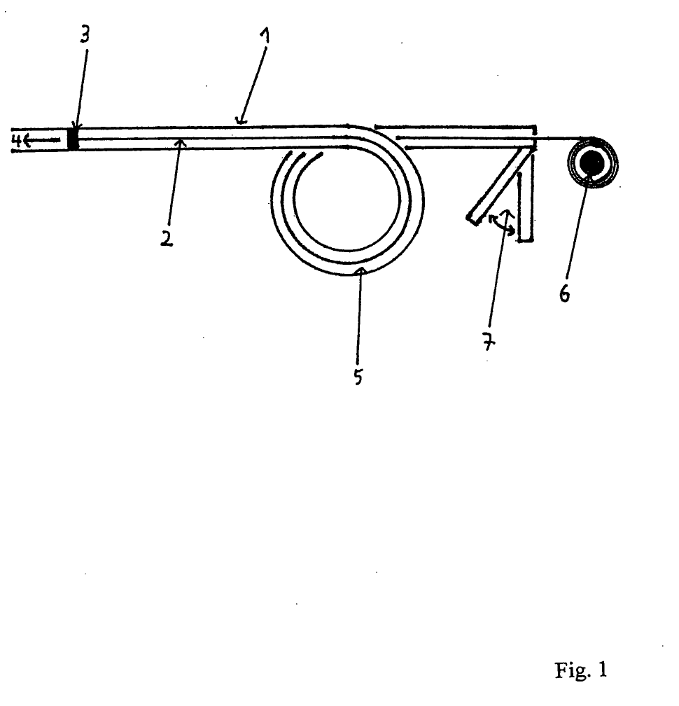

FIG. 1 is a schematic representation of the application device according to the invention. The application device comprises an application conduit 1 through which a movable, flexible piston rod 2 extends at which distal end a piston 3 is arranged. In the context of this invention the terms distal and proximal are to be understood such that proximal means, towards the physician's body when used according to the intended purpose” whereas the distal end is the one located away from the physician's body. Accordingly, the bone cement is injected from proximal to distal into the vertebroplasty cannula and finally into the vertebral body. As shown in FIG. 1 the distal end is situated to the left while the proximal end is on the right-hand side.

By actuating the piston rod 2 the piston 3 located at the distal end of the piston rod can be moved to and fro in the application conduit 1. It is of significance in this connection that piston 3 has sealing contact with the inner wall of the application conduit 1 for which purpose piston 3 is customarily provided with suitable sealing elements in its peripheral area. In order to inject bone cement into the vertebral body piston 3 must be moved in application conduit 1 in the direction of arrow 4 whereas by retracting piston 3 bone cement can be sucked into the application device.

In accordance with the invention the application conduit is at least partially wound up helically or spirally. In FIG. 1 a winding 5 can be seen; however, in the interest of representational clarity only a single winding 5 has been shown but it is to be noted that such an application device usually consists of several windings 5. The number of windings 5 to be provided for is thus governed by the total length of the application device necessary in relation to the useful inner length of application conduit 1 which in the first place depends on the volume or amount of bone cement required. The windings 5 may be arranged in the form of a helix or spiral, with a helix form in this context being a configuration in which the axis of the helix mainly corresponds with the longitudinal axis of the application device whereas in a spiral arrangement the axis of the spiral extends perpendicularly to the longitudinal axis of the application device. However, of importance to the invention is only that the application conduit basically extends at least partially in one or several windings while the respective arrangement of the windings 5 is irrelevant with respect to the basic functioning of the device.

At its proximal end piston rod 2 is wound up on reel 6 so that the entire application device can still be manipulated easily even if the piston rod 2 has been extracted almost completely from the application conduit 1 in proximal direction. Furthermore, the application device is provided with a handle 7 by means of which a ratchet mechanism is actuated causing the piston to be advanced in distal direction. The application conduit 1 in conjunction with piston rod 2 is customarily provided with a scale which enables the physician to monitor or observe the volume or amount of bone cement injected into the vertebra at a given time. By means of handle 7 the application device can be operated with one hand only so that handling the device is greatly facilitated.

1. Device for the application of bone cement, particularly in the field of vertebroplasty, comprising an application conduit (1) guiding the bone cement towards the application area and a flexible piston rod (2) longitudinally movable in the application conduit (1) and provided with a piston (3) arranged at the distal end of the rod (2), said piston being in contact with and providing a seal at the internal wall of the application conduit (1), with the piston rod (2) protruding proximally from said application conduit (1) characterized in that the application conduit (1) extends, at least partially, in one or several windings (5).

2. Application device according to claim 1, characterized in that the portion of the flexible piston rod (2) protruding from the application conduit (1) can be wound up on a reel (6).

3. Application device according to claim 2, characterized in that the piston (3) can be moved forward and backward within the application conduit (1) by turning the reel (6).

4. Application device according to claim 3, characterized in that the reel (6) is provided with a crank.

5. Application device according to claim 1 or 2, characterized in that the piston rod (2) can be moved forward and/or backward by means of a ratchet mechanism.

6. Application device according to any one of claims 1 to 5, characterized in that the piston rod (2) can be moved forward and/or backward by means of a motor.

7. Application device according to any one of claims 1 to 6, characterized in that the application conduit (1) has an inner diameter ranging between 2 and 5 mm.

8. Application device according to claim 7, characterized in that the application conduit (1) has an inner diameter ranging between 2.5 and 3 mm.

9. Application device according to any one of the claims 1 to 8, characterized in that the volume of the application conduit (1) that can be used for bone cement ranges between 4 and 10 ml.

10. Application device according to any one of the claims 1 to 9, characterized in that at its distal end the application conduit (1) is provided with connection capability for a cannula or needle.

11. Application device according to any one of claims 1 to 10, characterized in that the application conduit (1) is provided with a cooling system.

12. Application device according to claim 11, characterized in that the cooling system comprises one or several Peltier elements.
